(19) United States
(12) Patent Application Publication
(10) Pub. No.: US 2009/0306460 A1
(43) Pub. Date: Dec. 10, 2009

(54) Method and Apparatus for Controlling a Bodily Function

(75) Inventors: Anthony Clyde Neason Stephens, New South Wales (AU); John Furness, Victoria (AU)

Correspondence Address: JONES DAY, 222 EAST 41ST ST, NEW YORK, NY, 10017, US

(73) Assignee: Continence Control Systems International Pty Ltd., Chatswood, New South Wales (AU)

(21) Appl. No.: 11/990,493

(22) PCT Filed: Aug. 15, 2006
(86) PCT No.: PCT/AU2006/001158

§ 371 Date: Jun. 1, 2009

(30) Foreign Application Priority Data
Aug. 15, 2005 (AU) 2005904382

(51) Int. Cl. A61N 1/36

(52) U.S. Cl. 600/30; 607/41

(57) ABSTRACT

The present invention relates to a method and apparatus for controlling urinary incontinence. An implantable stimulator is utilised to apply stimulation to a sphincter or neosphincter about the urethra, in order to maintain coaptation of the urethra and urinary continence. Feedback is utilised in order to vary the level of stimulation applied to the sphincter in order to enable control over the sphincter. Control may be implemented by a user setting levels, by biofeedback, by a clinician.

### RELATED APPLICATIONS

U.S. Pat. No. 6,659,936, issued on 9 Dec. 2003, International Patent Application PCT/AU00/00925 filed on 4 Aug. 2000, Australian Provisional Application AU PQ2026, filed on 4 Aug. 1999, relate to controlling a bodily function. Each one of these documents are incorporated herein by reference in their entirety.

### FIELD OF THE INVENTION

The present invention relates generally to a method and apparatus for controlling stimulation of an anatomical feature in order to control a bodily function, and particularly, but not exclusively, to a method and apparatus for controlling stimulation of a sphincter for controlling incontinence.

### BACKGROUND OF THE INVENTION

Incontinence is a major medical problem. A number of treatments have been proposed, including stimulation of the bladder anatomy in order to maintain urinary continence.

It is known to use implanted sphincters and/or to stimulate the existing external urinary sphincter (EUS), in order to promote continence. It is also known to utilise a smooth muscle neosphincter (as disclosed in earlier International Patent Application No. PCT/AU00/00925, referenced above) which is implanted and stimulated by electrical signals in order to close the urethra.

Generally, with these prior art arrangements, the patient has no or limited control over operation of the closure of the bladder outlet. In the case of direct stimulation of the EUS, this may be less effective if there is underlying damage to the sphincter itself (for example when there is intrinsic sphincter deficiency). In the case of the smooth muscle neosphincter, for example, the patient is able to control whether the electrical stimulation is “on” (to close the urethra) or “off” to (to open the urethra to allow urination) without other unintended effects of stimulation, and no interim degree of closure depending on bladder state. By contrast, in the normal functioning bladder, a series of reflexes operate to allow filling, without overfilling and voluntary emptying, without unintended emptying, in a controlled and staged manner using feedback from mechanical sensors in and around the bladder (for example stretch receptors in the bladder wall or receptors that detect the presence of urine in the bladder outlet).

### SUMMARY OF THE INVENTION

In accordance with a first aspect, the present invention provides a method of controlling stimulation of implanted contractile tissue in order to control a bodily function, comprising the steps of determining the extent of stimulation required and varying the stimulation according to the determination.

In an embodiment, the anatomical feature is in the pelvic area for controlling a bodily function in the pelvic area.

In an embodiment the stimulation is electrical and is provided by a signal generator, the signal generator preferably being implanted in the patient's anatomy.

In an embodiment, the bodily function is bladder control, for maintaining urinary continence.

In an embodiment biofeedback is employed to determine the extent of stimulation required. Where the bodily function is bladder control, sensors may be required to determine bladder “fullness”. For example, a stretch or length sensor may be provided on the bladder to determine extent of fullness. The determination is then utilised to vary the stimulation applied. Another way of sensing bladder fullness that may be utilised is determining an electrical impedance via an impedance sensor between two points across the bladder. Other sensors may be used in addition or alternatively. This use of one or more sensors or other means to modify stimulation is called biofeedback.

In an embodiment, the step of determining the extent of stimulation required may be based on a time parameter. For example, monitoring a time period following a previous voiding of the bladder. In this embodiment, the method may include a further step of monitoring the physiology of the patient to determine the frequency of voiding of the bladder. A determination of the extent of stimulation required may then be based on determination of time periods since a preceding voiding event.

In an embodiment, the step of determining the extent of stimulation required may include the step of determining a setting of stimulation signal parameters of the stimulation device. In this embodiment, settings may be varied for different patient situations. For example, there may be a “day” setting and a “night” setting, which each result indifferent levels of stimulation being provided, minimizing the energy required by the stimulation system, providing variation in activation of the neosphincter and relieving any unnecessary pressure or tension on underlying tissue adjacent to the neosphincter. As another example, should the individual decide to participate in vigorous physical activity (for example a game of tennis, or sexual activity), the system may be temporarily configured to increase stimulation and reduce the likelihood or extent of leaks during this activity). A plurality of stimulation levels may be pre-set. In one example, there may be settings such as “off”, “low”, “medium”and “maximum”.

In an embodiment, the stimulation level may be variable.

The stimulation level may be varied under control of a user, who may be the patient.

In an embodiment, the stimulation levels may be set as part of a pre-calibration process implemented by a clinician.

In an embodiment, the bodily function is bladder control, and control is implemented by stimulation of a contractile tissue sphincter. The sphincter may be an existing urinary sphincter, and/or an implanted neosphincter. The implanted neosphincter may be a smooth muscle sphincter, such as described in PCT/AU2004/00925. Alternatively, the neosphincter may be implanted skeletal muscle. In a further alternative, an implanted smooth muscle neosphincter may be augmented with an implanted skeletal muscle sphincter. In a further embodiment, contractile tissue cells may be derived from another cell type, for example from progenitor cells or stem cells. Any appropriate contractile tissue may be utilised for the sphincter.

In one embodiment, the bodily function is bladder control, and control is implemented by stimulation of other features of the bladder anatomy.

In one embodiment, the user (or a supervising clinician) can enter additional data that characterises outcomes (for example, an event such as involuntary leak of urine, or as another example, if the patient feels they unnecessarily strain during the act of voiding) and the system uses this information, alone or in addition to patient-initiated voiding information (for example, when the system has been switched off temporarily) to adapt stimulation parameters to improve the system's performance.

Varying the stimulation preferably has the advantage of giving an ability to provide more optimum stimulation for bladder control. For example, in the invention disclosed in the above-referenced International patent application, where stimulation is provided to a smooth muscle neosphincter, a constant level of stimulation is provided until bladder voiding. The constant level of stimulation may be unnecessary. For example, when the bladder is emptied (just after voiding) it may not be necessary to stimulate, or a low level of stimulation only may be required.

In another example, applying stimulation after voiding, a relatively high level of stimulation may be applied immediately after voiding to cause a mucosal seal as quickly as possible. The stimulation may be set to a lower level once the seal has been established.

The ability to vary the stimulation, either based on biofeedback, user input to vary stimulation levels, or time parameter (eg time since urination), has the advantage of improving patient comfort and may also result in less urine leakage and in addition, prolong the battery life of the internal battery if one is incorporated in the implanted system.

In an embodiment, the step of determining the extent of stimulation required may be based on sensing a patient “event”, such as a change in abdominal pressure as a result of, for example a cough or a laugh or a change in posture, and modifying stimulation in order to compensate for the patient event. In an embodiment where a smooth muscle neosphincter is utilised, in accordance with the above-referenced PCT application, the same electrode or an additional electrode may be provided to provide an electrical signal to stimulate the EUS, to recruit the EUS to assist the smooth muscle sphincter to maintain mechanical seal of the bladder, in response to the patient event. Note that a patient event may include, as well as the above, an increase in physical activity, or a decrease in physical activity, a change in posture of the patient (e.g. the patient lying down, standing up) and any other activities of the patient.

In accordance with a second aspect, the present invention provides an apparatus for controlling stimulation of implanted contractile tissue in order to control a bodily function, the apparatus being arranged to determine the extent of stimulation required and to vary the stimulation according to the determination.

In accordance with a third aspect, the present invention comprises a computer program for controlling an implantable stimulator to control stimulation of implanted contractile tissue in order to control a bodily function, the computer program controlling the stimulator to determine the extent of stimulation required and to vary the stimulation according to the determination.

In accordance with a fourth aspect, the present invention provides a computer readable medium providing a computer program in accordance with the third aspect.

In accordance with a fifth aspect, the present invention provides a method of treating urinary incontinence, including the steps of applying an electrical signal to stimulate a neosphincter to maintain mechanical seal of the bladder and, in response to a patient event, the step of providing additional electrical stimulation to an area of the bladder anatomy to reinforce the closure of the bladder.

In an embodiment, the area of the bladder anatomy is the external urinary sphincter.

In an embodiment, the neosphincter is a smooth muscle sphincter implant as disclosed in the above-referenced PCT patent application.

Recruiting of another part of the bladder anatomy such as the EUS, in response to a patient event, advantageously supplements the resistance to leakage of the bladder which is provided by the stimulation of the neosphincter.

In accordance with a seventh aspect, the present invention provides an apparatus for treating urinary incontinence in a patient, the apparatus including a stimulator device including a signal generator arranged to provide an electrical signal for stimulation of a neosphincter to maintain a seal around the inner mucosa of the bladder, and stimulation of the part of the bladder anatomy to reinforce the closure of the bladder in response to a patient event.

In an embodiment, the apparatus includes a detector for detecting a patient event.

In an embodiment, the detector includes an abdominal pressure detector for detecting an increase in abdominal pressure.

In an embodiment, the part of the bladder anatomy is the EUS, and the apparatus includes an electrode arranged to be implanted in or adjacent to the EUS.

In accordance with an eighth aspect, the present invention provides a system including an apparatus in accordance with the sixth or seventh aspects.

In accordance with a ninth aspect, the present invention provides a system including a controller in accordance with the eighth aspect and an apparatus in accordance with the sixth or seventh aspects.

In accordance with a tenth aspect, the present invention provides a programmer enabling a clinician to interface with an apparatus in accordance with the sixth or seventh aspects of the present invention.

In accordance with an eleventh aspect, the present invention provides a system including a programmer in accordance with the tenth aspect and an apparatus in accordance with the sixth or seventh aspects.

In accordance with a twelfth aspect, the present invention provides a system including a programmer in accordance with the tenth aspect, a controller in accordance with the eighth aspect and a apparatus in accordance with the sixth or seventh aspects.

In accordance with a thirteenth aspect, the present invention provides a computer programme for implementing a controller in accordance with the eighth aspect.

In accordance with a fourteenth aspect, the present invention provides a computer readable medium including a controller in accordance with the thirteenth aspect.

In accordance with a fifteenth aspect, the present invention provides a computer programme for controlling a computing apparatus to implement a programme in accordance with the tenth aspect.

In accordance with a sixteenth aspect, the present invention provides a computer readable medium providing a computer programme in accordance with the fifteenth aspect.

In accordance with a seventeenth aspect, the present invention provides a method of controlling stimulation of augmented contractile tissue in order to control a bodily function, comprising the steps of determining the extent of stimulation required and varying the stimulation according to the determination.

### BRIEF DESCRIPTION OF THE DRAWINGS

Features and advantages of the present invention will become apparent from the following description of embodiments thereof, by way of example only, with reference to the accompanying drawings, in which:

FIGS. 1a and 1b are diagrams of the female and male bladder anatomy showing site of implant of a neosphincter;

FIG. 2 is a diagram of a female bladder anatomy illustrating an implanted stimulator device which is in accordance with an embodiment of the present invention;

FIG. 3 is a diagram of components of the stimulator of FIG. 2;

FIG. 4 is a diagram of an apparatus in accordance with an embodiment of the present invention;

FIG. 5 is a diagram of an apparatus in accordance with an embodiment of the present invention;

FIG. 6 is a flow diagram illustrating process steps in accordance with an embodiment of the present invention; and

FIG. 7 is a further flow diagram illustrating process steps in accordance with an embodiment of the present invention;

FIG. 8 is a further flow diagram illustrating process steps for pre-calibrating stimulation settings in accordance with an embodiment of the present invention;

FIG. 9 is a further diagram illustrating a process where the user directly selects the level of stimulation, in accordance with an embodiment of the present invention;

FIG. 10 is a further diagram illustrating user operation of settings, in accordance with an embodiment of the present invention;

FIG. 11 is a diagram illustrating a process of adjusting stimulation levels based on time since last micturition, in accordance with an embodiment of the present invention;

FIG. 12 is a diagram illustrating adjustment of stimulation levels based on sensor input, in accordance with an embodiment of the present invention

FIG. 13 is a diagram illustrating a process of optimisation of stimulator parameters, in accordance with an embodiment of the present invention, and

FIG. 14 is a diagram illustrating operation of yet a further embodiment of the present invention.

### DETAILED DESCRIPTION OF PREFERRED EMBODIMENTS

The following description of embodiments of the present invention will be given in relation to a method and apparatus for controlling urinary incontinence. The present invention-is not limited to the control of urinary incontinence, however. The method and apparatus in the present invention may be used for control of other bodily functions.

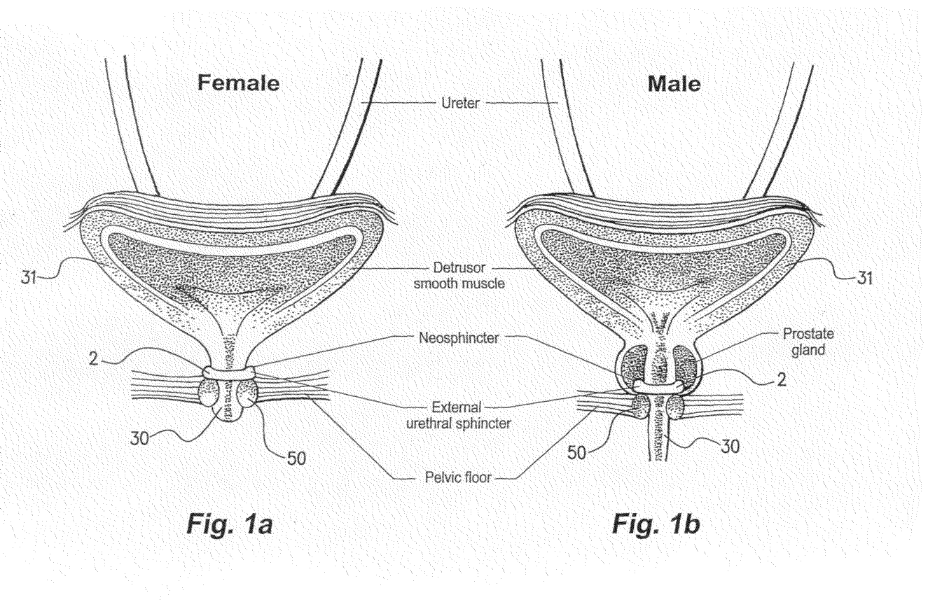

FIGS. 1a and 1b are diagrams showing aspects of the female and male urinary anatomy. The urethra in each Figure is denoted by reference numeral 30 and the bladder by reference numeral 31. A smooth muscle neosphincter 2 has been surgically implanted around the urethra 30 in a position close to the bladder 31 or other surgically convenient location. The sphincter 2 has been implanted as in accordance with the disclosure of the above-referenced PCT application.

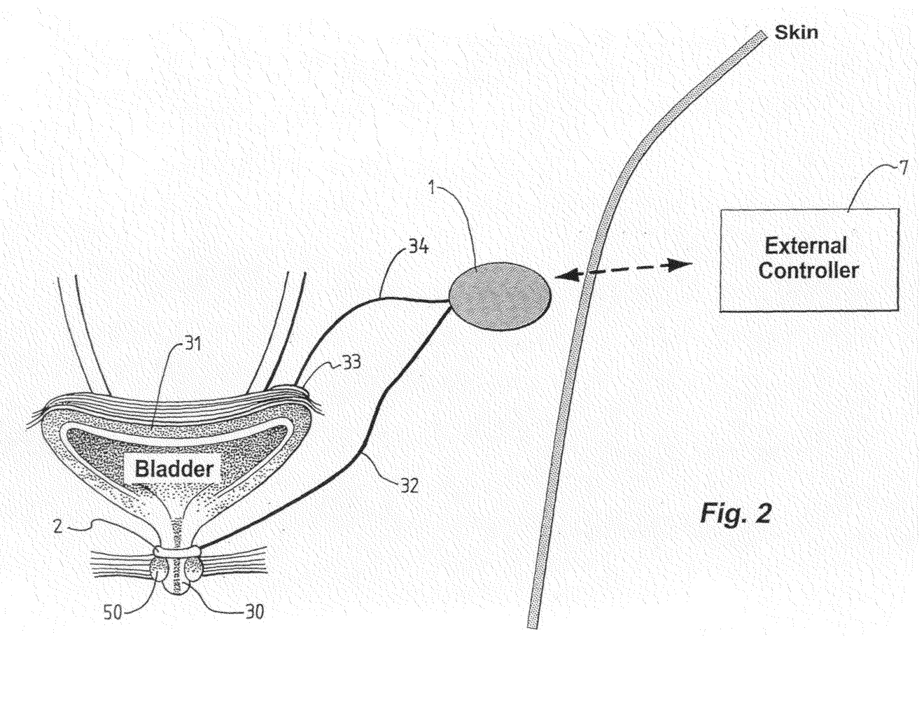

Referring to FIG. 2, which shows the female anatomy only (it will be appreciated the same arrangement can be transposed to the male anatomy), in accordance with an embodiment of the present invention, a stimulator device 1 has been implanted in the patient. The stimulator 1 may be implanted in any surgically convenient position, and in this embodiment is implanted between the abdominal muscles and the skin. The stimulator 1 includes a signal generator means arranged to provide an electrical stimulation signal for stimulating the smooth muscle sphincter 2. A lead 32 extends from the stimulator 1 to an electrode at the neosphincter 2, for providing the stimulation signal to the neosphincter.

The electrical stimulation signal provided by the signal generator maintains contraction of the smooth muscle sphincter 2, in order to prevent urine flow through the urethra and therefore maintain continence. The stimulation signal may be a charge balanced biphasic pulse of predetermined frequency and predetermined amplitude. Any other convenient signal shape may be utilised.

In accordance with the system disclosed in the above-referenced PCT application, the stimulator 1 may also be arranged to produce a further electrical signal to stimulate the sphincter 2 to relax, to allow urine to flow through the urethra and enable the patient to evacuate their bladder. Instead of a further electrical signal, the stimulator 1 may be arranged to stop producing any electrical signal and it is the absence of a signal that causes the sphincter to relax.

In accordance with an embodiment of the present invention, the stimulator also incorporates an arrangement for determining the extent of stimulation that may be required, and for varying the stimulation according to the determination. The determination may be made in a number ways.

In FIG. 2 a stretch sensor 33 is implanted on the external surface of the bladder 31. Signals from the stretch sensor 33 can be used to determine the “fullness” of the bladder. These signals are fed back via a further lead 34 to the simulator 1 to control the stimulation signal applied to neosphincter 2. When the bladder is not full, the stimulation signal may be varied so as not to provide as much stimulation to the neosphincter 2. The signal may be increased as the bladder fills (as the stretch receptor provides an indication that the bladder is filling).

The stimulation signal may be varied by varying the frequency of the signal and/or the amplitude of the signal.

Use of a stretch receptor to determine bladder fullness is one form only of biofeedback that may be used to make the determination as to the extent of stimulation required. Other forms may include monitoring of intravesical pressure or volume, wetness or ammonia, utilising different sensors. A combination of sensors may be utilised.

Other sensors may assess bladder fullness by measuring the impedance to detect fluid in the bladder, by sensing bladder pressure, presence of urine in the bladder neck, deformation of the urethra, and other approaches.

Various sensor approaches may include the following:
Detecting the electrical impedance between two points across the bladder, using an impedance sensor. The urine in the bladder may change the path length/bulk impedance.
A sensor element for detecting changes in abdominal pressure. In an embodiment this may include a piezoelectric element which generates different electrical signals during changes in abdominal pressure by breathing and/or stress events when the bladder is emptied compared with full.
Use of circumferential or a backing mesh support to a neosphincter placed around or in the vicinity of the urethra that senses dilatational deflection in the presence of an urge or stress event. Again a piezoelectric element may be employed within the body of the supporting element.
A dedicated sensor may be placed in the vicinity of the bladder wall to detect changes in abdominal pressure (a full bladder may be less compliant when subjected to sudden pressure change when compared with an empty bladder).
Detection of sudden movements of the patient may also be sensed. This may be done by detecting a sudden movement of the stimulator sutured to the abdominal wall. Any physical movement may be detected. For example, urge events may be detected if there is a sudden straining with the system still switched on.
A piezo-electric element mounted on or proximate to the urethra may generate a signal when the piezo-electric element is strained, due to swelling of the urethra by an unwanted leak to act as a trigger sensor to increase stimulation unless the stimulation level is “off” (i.e. the patient wishes to void).

More than one sensor or combinations of different types of sensors may be utilised.

In an embodiment a sensor or sensors may be used to sense if bladder pressure rises too high (which may risk renal reflux) with the stimulator switched on. If this detected pressure is achieved, in this embodiment the stimulator is arranged to automatically lower the stimulus to prevent renal damage, and also maintain some stimulus to limit the severity of a leak.

In another embodiment, to be described later in relation to FIG. 6, no biofeedback sensors may be employed, but instead the stimulator 1 is arranged to vary stimulation in accordance with a predetermined algorithm. The predetermined algorithm may be based on “learning” the physiology of the patient by the system, in order to, for example, determine the usual frequency between episodes of micturition, and to vary stimulation such that, for example, immediately after evacuation the stimulation signal is less than later after evacuation.

In another embodiment, stimulator 1 may be provided with one or more “settings”, and the determination of the extent of stimulation required is made based on the setting. In one embodiment, there may be a setting for “night” operation and a setting for “day” operation. The night operation may require less signal stimulation than the daytime operation. This embodiment will be described in more detail in relation to FIG. 7. Other embodiments which include different ranges of settings are described with reference to some of the other figures.

In another embodiment, a sensor is arranged to detect a sudden change in abdominal pressure, chest volume (cough, sneeze) or movement (jump, jolt). The detected patient “event” temporarily modifies stimulation to augment closure force. For example, stimulation may be increased from Medium to High. A sensor may also detect a change in position of the patient. For example it may detect that a patient is lying down and reduce the level of stimulation.

In another embodiment, stimulation may also be provided to other anatomical features. For example, stimulation may be applied to the external urinary sphincter (EUS) Where a neosphincter is implanted about the urethra, stimulation may be varied by applying stimulation to the EUS as well as to the neo-sphincter.

The embodiments discussed above may be used separately or together. For example, stimulation may be based on a “learned” algorithm used together with biofeedback to provide control of the stimulation signal. User input may also be implemented to move between a plurality of stimulator settings.

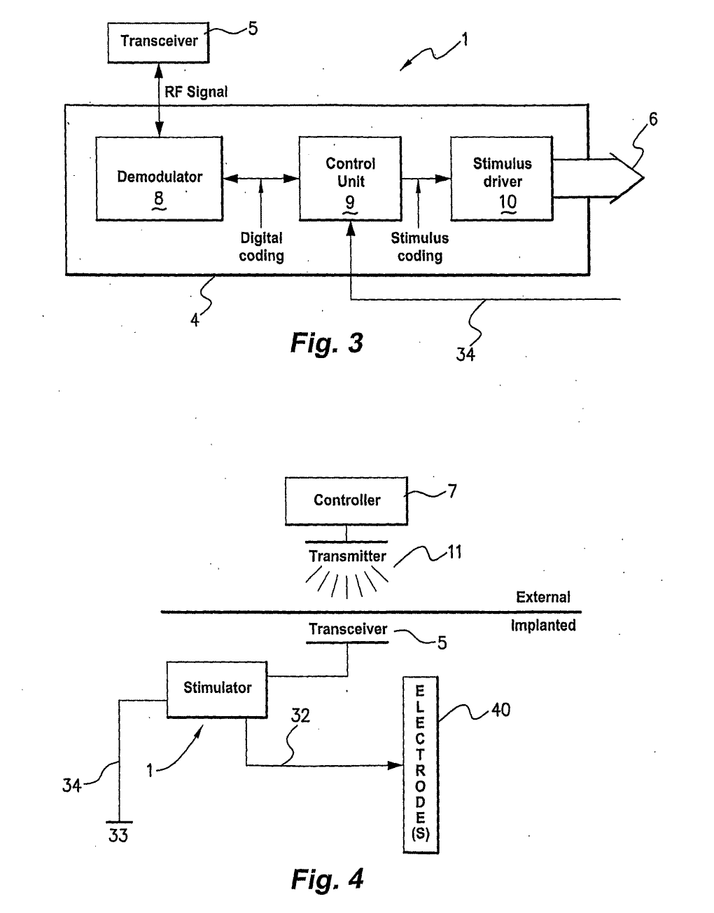

The stimulator 1 is shown in more detail in FIG. 3. In this embodiment, a signal generator arranged to provide the electrical signal for stimulation of the sphincter is in the form of a control unit 9 and stimulus driver 10. The control unit 9 encodes the stimulus and provides a signal to the stimulus driver 10 which provides the stimulation signal at output 6. As discussed above, the output 6 outputs to conductor 32 and to one or more electrodes.

The control unit 9 is also arranged to receive a signal from lead 34 connected to sensor 33 on the bladder 31. The sensor may be placed in another position if it can still detect bladder fullness. More than one sensor may be used in different types of sensors as discussed previously. The control unit 9 processes the signal from the lead 34 to determine the extent of “fullness” of the bladder. Depending upon the determined “fullness”, the control unit then controls the stimulus driver to provide a stimulation signal of a predetermined level. The less full the bladder, the lower the level of the stimulation signal that will be provided. The control unit may be programmed with a number of predetermined signal levels eg signal level 1 (low strength), signal level 2 (medium strength) and signal level 3 (full strength) These signal levels will be selected between depending upon the signal from the stretch receptor 33. In other embodiments, more levels may be provided or the signal may be varied in an analogue fashion.

In this embodiment, the control unit 9 and stimulus driver 10 form, together with a demodulator 8, a processing unit for generating the stimulation signal(s) at output 6.

The demodulator 8 is arranged to demodulate a signal received by transceiver 5. An external control unit and external programmer unit are able to communicate via the transceiver with the processing unit 4 in order to control application of stimuli and/or vary the stimuli. In addition, as described in more detail later, the processing unit 4 may transmit, via control unit 9, demodulator 8 and transceiver 5, signals to the control unit or programmer unit. The transmitted signals may deliver telemetry information indicative of parameters of the stimulator, the information may be used for many purposes, including, but not limited to the purposes of calibration and control. In an example, the transmitted signals may provide information that can be used for clinician monitoring of the patient.

The entire stimulator 1 (including components 4 and 5), is enclosed in a housing which includes a casing made from a bio-compatible material, such as titanium, silicone rubber or other acceptable materials, or combinations of materials, including, but not limited to inert materials. The frequency of the RF signal for transmission and reception by the transceiver 5 may depend on the material of the casing of the stimulator.

FIG. 4 shows an apparatus in accordance with an embodiment of the present invention. The apparatus incorporates the implanted stimulator 1, with transceiver 5. The electrode(s) 40 is shown schematically together with cables 32, 34 and sensor 33.

The apparatus also comprises an external controller 7 which includes a transmitter 11. The controller 7 is intended for operation by a patient with the stimulator implanted, for control of the stimulator 1.

The controller 7 includes means (such as a button,or any other acceptable way, not shown) operable by the patient to selectively send signals to the implanted stimulator 1, for control of the stimulation signals being sent to the electrodes 40. In this embodiment, the stimulator is “fail safe”. Unless a signal is received from the controller 7, the stimulator produces a signal which maintains tone in the smooth muscle implant 2, maintaining pressure on the urethra.

When the patient wishes to urinate, they actuate the controller 7 to send, via the transmitter 11, a signal to the stimulator. In response to receiving the signal, the control unit 9 operates to turn the stimulating signal off causing the sphincter to relax and allow the patient to urinate.

The controller 7 may also be arranged to provide a further signal under patient control, once the patient has finished urinating, the further signal causing stimulator 1 to resume providing the stimulation signals to the electrode(s) 40. In another embodiment (described later) user input may control the stimulator to move between a plurality of stimulation levels.

In “fail safe” mode, if the further signal is not produced, the stimulator will resume providing the stimulation signal to the electrodes 40 after a pre-determined period of time.

The stimulation signal 6 provided to contract the smooth muscle sphincter 2 is selected so as to provide a substantially continuous contractile activity in the sphincter. A charge-balanced biphasic pulse may be suitable for this. The signal has a substantially constant current less than or equal to 30 mA, and may be in the order of 15 mA. Stimulation pulse frequency provided to sphincter 1 is in the range of 0.25 Hz to 5 Hz and is preferably 2 Hz. Stimulation pulse width is in the range of 0.05 milliseconds (ms) to 1 ms and is preferably 0.15 ms. These parameter values will generally be for the case where “full” signal is being provided to maintain tone in the sphincter. The values will be less where less stimulus is required eg in the event of biofeedback determining that less stimulation is required. The stimulator is current regulated, and accordingly the stimulation voltage will vary with the resistance of the muscle tissue between the electrodes. Typical values for the voltage may be between 0.2 and 7 volts.

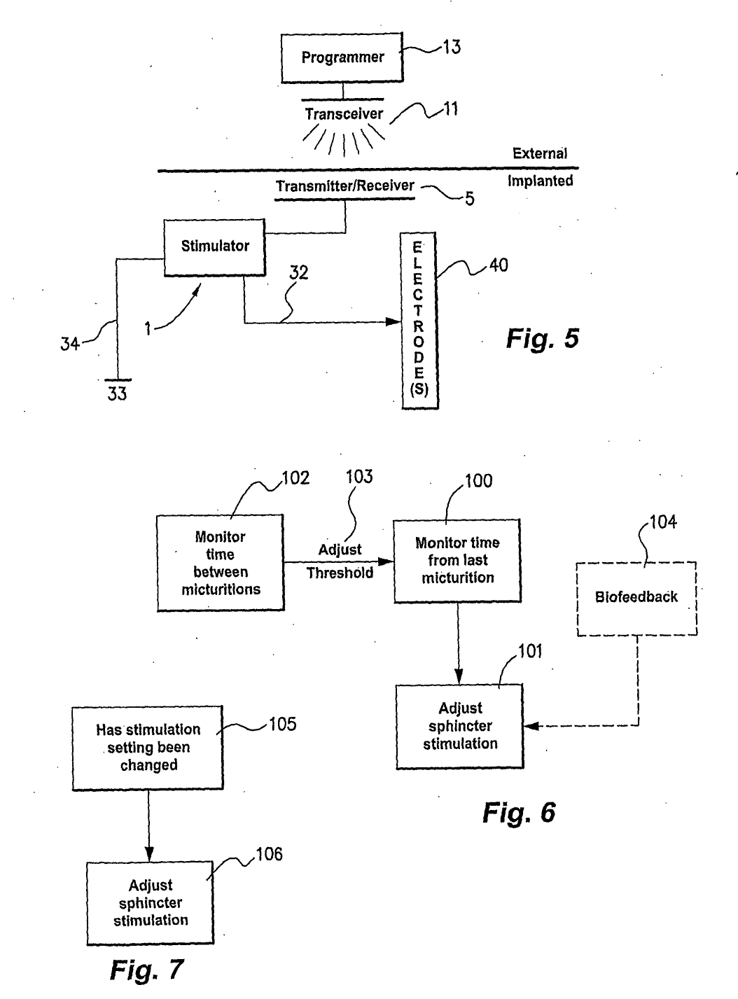

FIG. 5 shows an apparatus in accordance with an embodiment of the present invention, including a programmer unit 13 which may be utilised by a physician to set and adjust parameters of the implanted stimulator 1. The programmer unit 13 may be an appropriate device for communicating with the stimulator via transceiver 11, and may include a computing device. The control unit 9 is also arranged to transmit stimulator telemetry information indicative of one or more of the parameters of the stimulator 1, for detection by the programmer 13 via transceiver 11. The programmer unit 13 can therefore determine parameters of the stimulator from telemetry information and can adjust the parameters by transmitting control signals to the stimulator 1. The signal from the programmer may be able to selectively vary the output current, shape, frequency and/or pulse width of the stimulation signal(s).

In operation, a physician may adjust parameters of the stimulation signal(s). The physician will note feedback from the patient as to the effect of the stimulus on bladder control, and may subsequently re-adjust the parameters until the stimulation is optimum. For example, patient perceived feedback may be used to set the maximum stimulation threshold of the smooth muscle sphincter (for example, any overflow stimulation to the neosphincter may elicit and/or be perceived as an urgency event by the patient).

In the above-described embodiments, signals between the controller or programmer and the stimulator are RF signals. Other types of transmission media other than RF may be used. For example, microwave signals may be used for transmission, optical signals may be used, and in another embodiment magnetic transmission may be used.

Magnetic transmission may be used for the controller unit 7 to cause the stimulator to stop producing stimulation signals and therefore allow the patient to urinate. In this embodiment, the control unit 7 may be a simple magnet which, when passed over a magnetic receiver of the stimulator 1, results in the stimulator ceasing to provide stimulation signals for contracting the sphincter. Other means than magnetic transmission may be utilised, such as optical, microwave signals and others.

FIG. 6 is a diagram illustrating a process for determining the amount of stimulation required for stimulation of the sphincter 2 and for adjusting the sphincter stimulation 2. This process does not require biofeedback. Instead, sphincter stimulation is adjusted based on a perceived micturition need. In this embodiment, the determination of the need for micturition is based on monitoring a time parameter, in this case being the time from the last micturition (step 100). If that time is over a predetermined threshold, then sphincter stimulation is adjusted (step 101). It is assumed that if a person has just urinated then their bladder will be empty and stimulation level required the will not be high. The sphincter may therefore be stimulated with a relatively low signal, or even not at all. After a time period, it will be assumed that the bladder has started to fill and that adjustment to the sphincter stimulation is required in order to ensure that continence is maintained.

The process may involve a number of different thresholds so that there are a number of different levels of stimulation applied to the sphincter, dependent upon time from the last micturition.

In an enhancement of this embodiment, a further step in the process is provided which monitors the time between micturitions and “learns” an average time between micturitions which can be used to adjust the threshold (step 103) for adjusting the sphincter stimulation. This is in fact a form of biofeedback which does not require a sensor, merely knowledge of frequency of urination (the control unit 9 will obtain this knowledge via knowledge of each time the stimulation is turned off by the user to allow for micturition).

Note that the control unit 9 may be appropriately programmed to implement the process of FIG. 6. No lead 34 or sensor 33 may be required.

In a further embodiment, however, biofeedback 104 may be utilised to determine adjustment to sphincter stimulation 101 in addition to process step 100. In that case, the biofeedback may include a sensor 33 and lead 34, as discussed above.

FIG. 7 illustrates a further process for adjusting the sphincter stimulation. This process depends upon determining whether a setting of the stimulator 1 has been altered. Stimulator settings are available for operation by the user. For example, one setting may be available through day time and another for night time. This may be used for, example, where no stimulation is ideal at night time in order to “rest” the sphincter. In that case the patient at night time may wear other protective devices to deal with the effects of incontinence.

Alternatively, a setting may be utilized to provide stronger stimulation, for example when performing vigorous activities that may yield in a higher frequency or severity of stress incontinence episodes and a lighter stimulation while at rest.

The embodiment of FIG. 7 may be used with the embodiment of FIG. 6 and the different settings could include a setting which enables the embodiment of FIG. 6 or disables the embodiment of FIG. 6.

The embodiment of FIG. 7 may also be used with biofeedback.

As discussed above, the stimulator may include a range of stimulation settings. In a further embodiment, the settings may be as follows:
“off” (patient selectable voiding setting). No stimulation.
“low”. Minimum frequency, minimum stimulus intensity to cause coaptation of the urethra with sub-maximal bladder fullness/bladder pressure.
“medium”. Minimum frequency, moderate/maximum intensity to cause coaptation of the urethra.
“maximum”. Maximum frequency, maximum intensity to provide additional closing effect on the sphincter, without enlisting unwanted side effects. This setting may be used, for example, where the patient is undertaking highly active physical actions, during sex, for example, or partaking in a sport.
“variable”. The settings are configured to allow either a variety of discrete settings or just a continuous variation of the settings.

The actual values for the range of stimulation settings may depend upon variables such as the patient's physiology, the nature of the contractile tissues sphincter (and its size), the componentry used to implement the stimulator, and other factors. Generally, however, the stimulator settings will be set to produce the above bullet pointed effects.

The stimulator has a number of parameters that may be varied to vary the stimulation setting, including current, frequency and pulse width. It should be understood that these parameters each have an effect and that different combinations of values of these parameters may be used to provide Low, Medium and Maximum stimulation settings. In one example, the following ranges may be utilised for stimulator values to provide the stimulation settings:
Threshold (Threshold an effect is just being felt and below Threshold is Off) 1 mA to 3 mA, 0.4 ms to 0.6 ms and 0.5 Hz to 1.5 Hz, in one embodiment 2 mA, 0.5 ms, 1 Hz.
Low: 3 mA to 5 mA, 0.4 ms to 0.6 mS, 0.5 Hz to 1.5 HZ, and in one embodiment 4 mA, 0.5 ms, 1 Hz.
Medium: 6 mA to 10 mA, 0.4 ms to 0.6 ms, 0.5 Hz to 1.5 Hz, and in one embodiment 8 mA, 0.5 ms, 1 Hz.
High: 6 mA-10 mA, 0.4 ms to 0.6 ms, 1.5 Hz to 2.5 Hz, and in one embodiment 8 mA, 0.5 ms, 2 Hz.

It should be noted that to preserve battery life, it is better to increase stimulus intensity (current) first, then increase stimulus frequency.

Further, increase phase width may help the stimulation efficiency, so, for example, if the threshold is at 5 mA, the clinician may try a wider phase width (e.g. 1 ms) and the threshold may be increased to 4 mA, creating a bit more “headroom” for the other stimulation settings. All allowable parameters must fit within the parameter space of the stimulator's capabilities.

These stimulation settings may be utilised in a number of ways.
1. User selection—based on desired or observed outcomes. The patient selects between stimulator settings based on desired frequency of urination and/or observed leaks. The controller unit 7 is provided with enough user input user means (for example in the form of user actuated buttons) to enable the user to select between settings.
2. Operation Environment mode selected by the user. These include a “day” mode which is equivalent to the Medium setting. A Night mode which is equivalent to the Low setting. An Active mode which is equivalent to the Maximum setting, for example, for more intense physical activity.
3. Movement between stimulator settings may be determined based on times since micturition. After a patient initiated voiding event, the system returns to Low, then at a programme time, automatically increases to a Medium setting assuming that the bladder now has more fullness. When a urination event occurs, the cycle can repeat.
4. Movement between stimulator settings may be initiated based on feedback from sensors. The sensor input of bladder for stimulation setting between Low Medium and Maximum depending on what is being sensed. Maximum stimulation may be used if the sensor indicates that the risk of leak is high for example a sensor that may detect that the patient is undertaking physical activity (i.e. the sensor senses sudden movements).
5. Treatment Evolution. As the patient's bladder physiology changes (for example bladder compliance improves, given bladder is now filling rather than gradually leaking, or there are changes in the dynamics of the urethra (becoming stiffer or, alternatively, more compliant) different control of stimulator settings may be required. For example, the patient may be in the Maximum mode more often. This may require the clinician to use the programmer unit to change the relevant stimulus paramount parameter sets. Alternatively, at follow-up the clinician could observe the duration of time spent by the patient at each of the settings. For example, where a patient is on Maximum too often to achieve the patient's desired outcome, the clinician could provide feedback on what should be the appropriate parameters (for example increasing the stimulus setting corresponding to Medium).
6. Immediately after a patient voiding event, the system may set stimulation to Maximum automatically in order to cause mucosal seal as quickly as possible after voiding, then backing off the stimulation to a lower setting, such as Medium. A separate stimulation setting for causing of coaptation may be included in an alternative embodiment which is used for a short period of time (for example 5 to 60 seconds) after voiding (i.e. not Low, Medium or Maximum settings, but a separate setting for Coaptation).

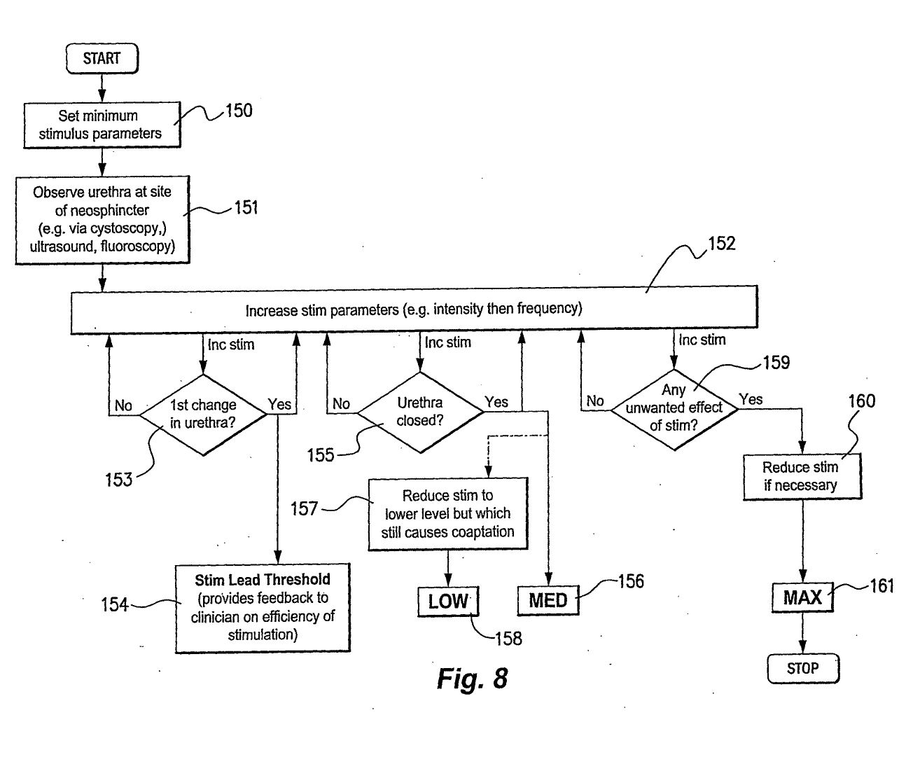

In order to establish settings, in an embodiment a pre-calibration process is implemented, for example by a clinician. FIG. 8 is a flow diagram illustrating a pre-calibration process in accordance with one embodiment of the present invention. In order to set the stimulator settings 150 the clinician observes the urethra at the site of the neosphincter 151 (for example by cystoscopy, ultrasound, through ostopy etc) and increases the stimulation settings (for example intensity then frequency) 152 while continuing to observe the urethra 151. On observation 153 of the first change in the urethra the clinician sets a Stimulation Lead Threshold 154 which provides feedback to the clinician on the efficiency of stimulation. On observation 155 that the urethra is closed the clinician sets the Medium 156 setting. Subsequently the clinician reduces 157 the stimulator setting to a lower level which still causes coaptation and sets 158 this level at the Low level.

The clinician then increases the stimulation level and observes 159 any unwanted effect of the stimulation, such as pain or irritation, then backs off 160 the stimulation level to stop the unwanted side effect and sets this level as Maximum 161.

It will be appreciated that other pre-calibration processes can be used to set further levels or alternative levels. The patient may visit the clinician periodically to re-set stimulation levels.

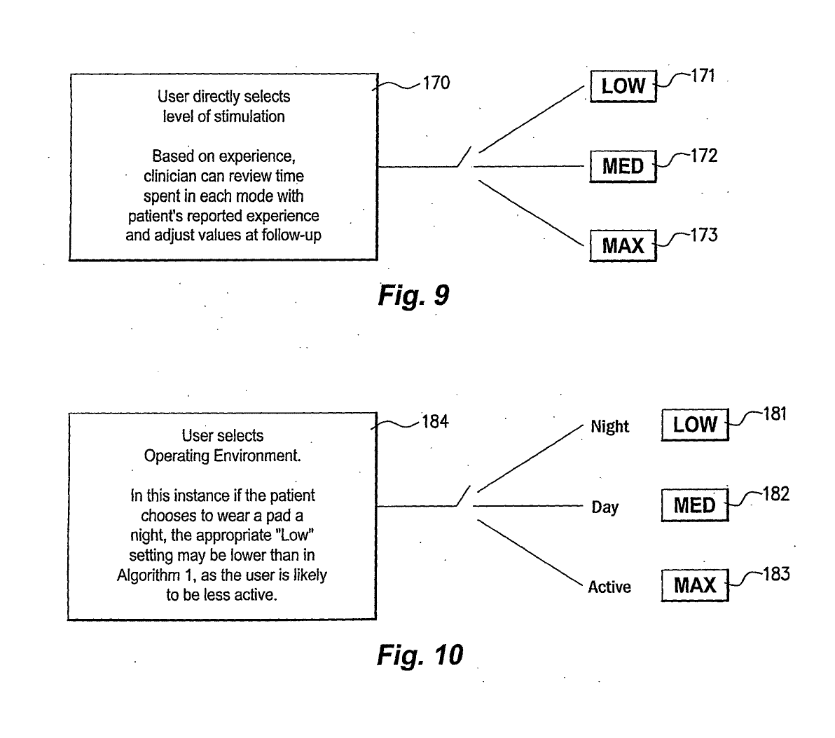

FIG. 9 is a diagram illustrating that a user can directly select 170 their level of stimulation if an appropriate controller unit is provided. The user selects between Low, Medium and Maximum 171, 172, 173. The clinician may review the time spent in each mode with patients recorded experience and adjust at a follow up visit. In this embodiment, the controller includes the user actuator usable interface (e.g. a rotary switch or other type of switch or control) to enable the user to switch between stimulation and settings.

FIG. 10 illustrates that the levels Low, Medium and Maximum, may be used synonymously with Night and Day and Active operating environment modes 181, 182, 183. Again, the user selects 184 between the levels in this particular operating environment. The patient may choose to wear a pad at night and the appropriate Low setting may be lower than in the process illustrated in FIG. 8, as the user is likely to be less active.

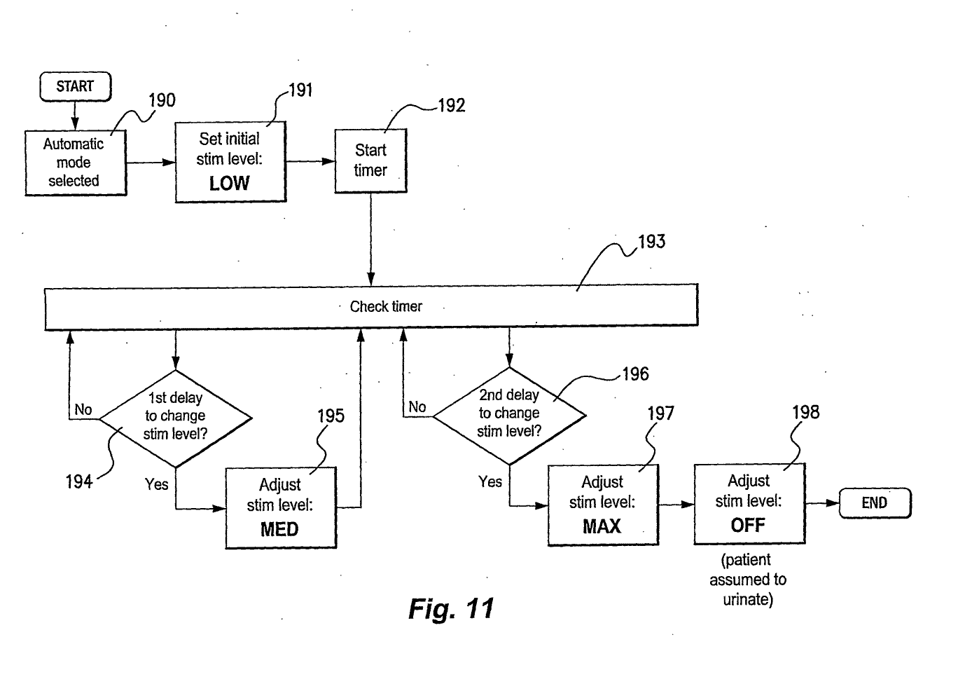

FIG. 11 is a flow diagram showing a more detailed process for automatic adjustment between the set levels based on times since last micturition (described above in relation to FIG. 6). The system may have an “Automatic” mode which may be selected 190 by a patient (with an appropriate control unit) or selected by the clinician. An initial stimulation level of Low is set 191 after a patient voiding event. Note that in an alternative embodiment, an initial higher level may be implemented for a predetermined time in order to cause coaptation of the urethra before moving back to the Low level. A timer 192 is started to time delay periods. The timer is checked 193 by the system to establish whether the delays have yet passed. When the first delay has passed 194 the stimulator level is adjusted 195 to Medium. When a second delay has passed 196 the stimulation level is adjusted 197 to Maximum. When the patient wishes to void, they adjust 198 the stimulation level to Off. The cycle then repeats.

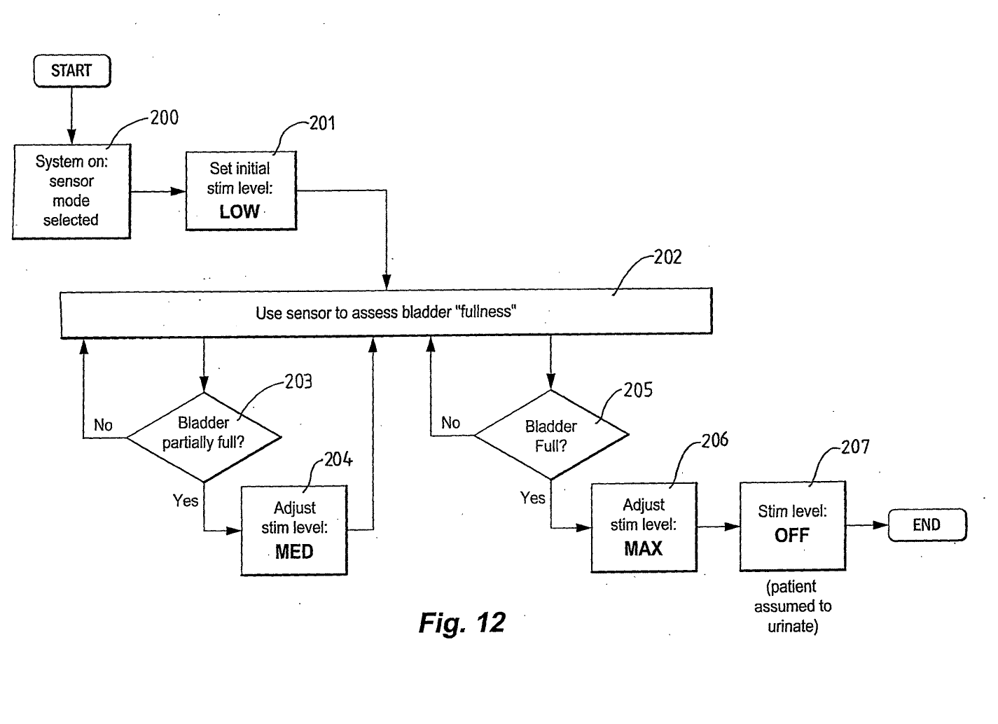

FIG. 12 illustrates a process of adjusting the stimulator settings based on feedback sensed by a sensor(s). A sensor mode may be selected 200 either by a patient with an appropriate control unit or by the clinician on calibrating/programming of the system. The initial stimulation is set 201 to Low and then the sensor is utilised to assess bladder fullness 202. If the bladder is partially full 203 stimulation is adjusted 204 to Medium. If the sensor senses that the bladder is full 205, the stimulation is adjusted to Maximum 206. When the patient urinates (stimulation level Off 207) the cycle repeats. Note that, as discussed above, many different sensors may be used and not just to assess bladder fullness. Sensors may be used to assess physical activity, for example, and stimulation settings adjusted accordingly.

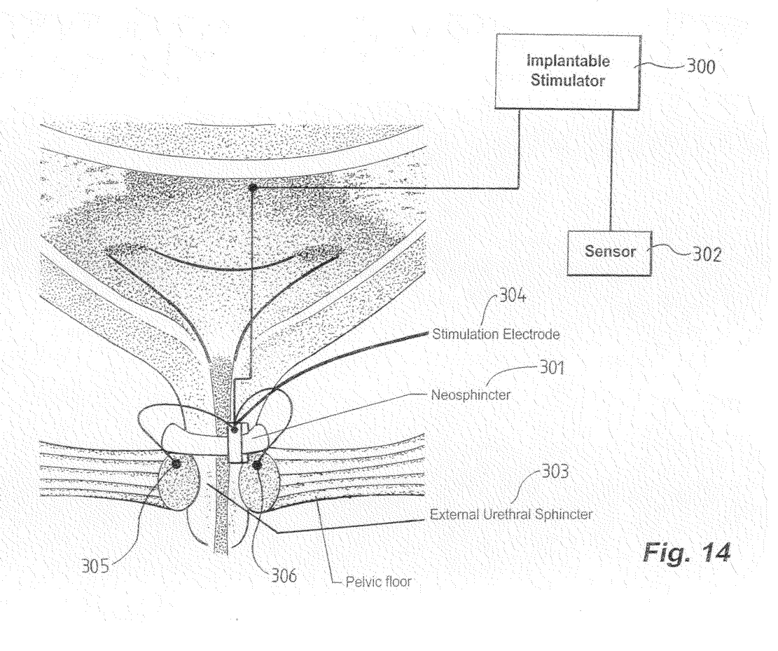

FIG. 14 illustrates a system in accordance with the further embodiment of the present invention. This utilises an implantable stimulator 300 that can modify stimulation to augment a neosphincter 301 and reduce the severity of leakage. A sensor 302 is arranged to detect a sudden change in abdominal pressure, chest volume (cough, sneeze) or movement (jump, jolt). The detected patient, “event” temporarily modifies stimulation to the EUS 303 to augment closure force. This embodiment shows a electrode 304 for stimulating the neosphincter 301 and further electrodes 305, 306 for stimulating the EUS 303. While the EUS may provide additional closure, other pelvic anatomy such as other muscles of the pelvic floor may also assist preventing leakage of urine in combination with the neosphincter. Further electrodes may be provided in appropriate parts of the pelvic floor to utilise the pelvic floor to assist the neosphincter in maintaining continence.

In a further embodiment a sensor is provided to sense when a person is in a horizontal position “lying down” or upright. Sensors can be incorporated within the housing of the stimulator 300. On lying down (eg to sleep) the stimulator 300 will first sense the horizontal position and may change one or more parameters of the stimulation signal.

The stimulation may be altered in accordance with any patient event and/or other anatomical features such as the EUS may be recruited to assist. For example, a change in pressure caused by coughing or laughing may result in an increase in stimulation to a neosphincter and/or to the EUS.

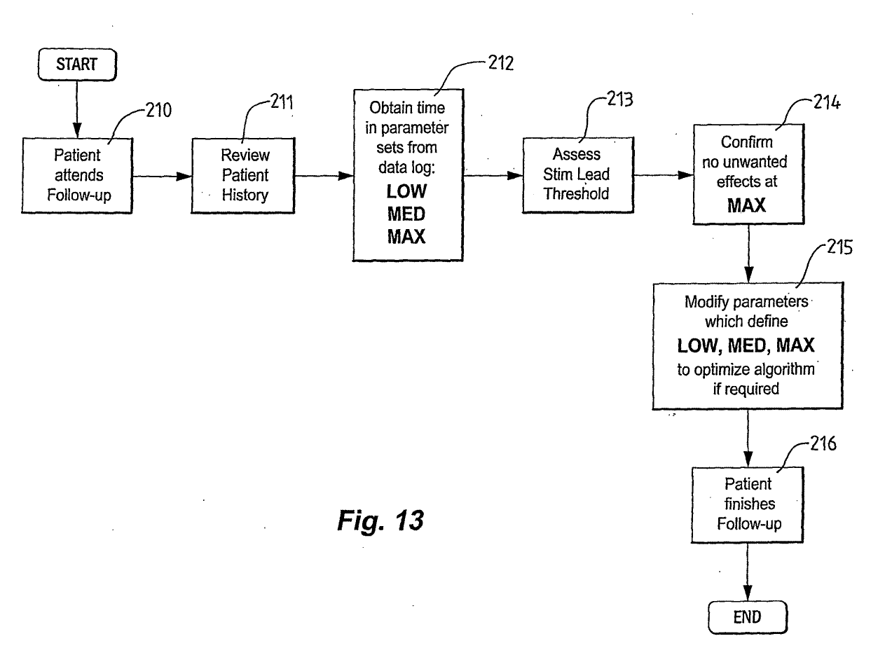

FIG. 13 illustrates one process that might be implemented for patient follow ups. In this process, the patient attends 210 a follow up visit at the clinician. The patient's history is reviewed 211 and it is determined what amount of time the patient has spent previously in parameter settings Low, Medium and Maximum 212. In this embodiment, the stimulator control unit includes a memory which includes a data log showing these times. A Stimulation Lead Threshold is assessed 213 and a recalibration to confirm no unwanted effects at Maximum setting 214 are carried out. Parameters for Low, Medium and Maximum may be adjusted 215. The patient follow up is finished 216.

The apparatus of the above embodiment provides stimulation signals to a neosphincter in the form of a smooth muscle sphincter which is usually taken from elsewhere in the body and transplanted around the urethra, as taught in International Patent Application publication no. WO 01/10357. The apparatus of the present invention is not limited to providing stimulation signals to such a neosphincter. Signals may instead be provided to other anatomical features. For example, the external urinary sphincter may be stimulated to exert pressure on the urethra, in the absence of any smooth muscle neosphincter. Where a neosphincter or other implanted sphincter is present, stimulation may be varied to the neosphincter and also applied in addition to the EUS in order to supplement the force exerted on the urethra.

In the above embodiments, power sources for the implantable stimulator will be provided in the form of batteries. These are not shown in the diagrams. They may be primary cells that are depleted (requiring replacement of the stimulator), rechargeable or the implant may be powered by RF or other forms of energy transmitted from outside the patient.

As discussed above, the stimulator implant is preferably sealed and encased in a biologically inert material such as a bio-compatible silicone material. Metallic electrodes and leads are preferably of material that is biologically inert (e.g. platinum or a platinum-iridium alloy). The connecting wires are preferably placed between the abdominal muscle and the skin.

In the above embodiments, software may be controlled to hardware to implement the methods and apparatus of the invention. It will be appreciated that the software may be designed to perform the functions discussed in this specification.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It will be appreciated by persons skilled in the art that numerous variations and/or modification may be made to the invention as shown in the specific embodiments without departing from the spirit or scope of the invention as broadly described. The present embodiments are, therefore, to be considered in all respects as illustrative and not restrictive.

1. A method of controlling stimulation of implanted contractile tissue in order to control a bodily function, comprising the steps of determining the extent of stimulation required and varying the stimulation according to the determination.

2. A method in accordance with claim 1, wherein the bodily function is associated with continence.

3. A method in accordance with claim 2, wherein the bodily function is bladder control, for maintaining urinary continence.

4. A method in accordance with claim 3, wherein biofeedback is employed to determine the extent of stimulation required.

5. A method in accordance with claim 4, wherein the biofeedback is arranged to provide an indication of bladder fullness.

6. A method in accordance with claim 4, wherein biofeedback is implemented by sensing stretch of the bladder wall.

7. A method in accordance with claim 4, wherein biofeedback is implemented by sensing electrical impedance associated with the bladder.

8. A method in accordance with claim 4, wherein the biofeedback is implemented by sensing deformation of the urethra and/or bladder.

9. A method in accordance with claim 4, the biofeedback being implemented by sensing bladder pressure.

10. A method in accordance with claim 4, the biofeedback being implemented by sensing presence of urine in the bladder neck.

11. A method in accordance with claim 4, the biofeedback being implemented by sensing physical activity of a patient.

12. A method in accordance with claim 11, the step of sensing physical activity comprising the step of sensing sudden movements of the patient.

13. A method in accordance with claim 3, wherein a time parameter is employed to determine the extent of stimulation required.

14. A method in accordance with claim 13 wherein the time parameter is a time period following previous voiding of the bladder.

15. A method in accordance with claim 13, comprising a further step of monitoring the patient to determine the frequency of voiding of the bladder.

16. A method in accordance with claim 15, wherein the determined frequency of voiding of the bladder is employed in the step of determining the extent of stimulation required.

17. A method in accordance with claim 3, wherein user input is employed to determine the extent of stimulation required.

18. A method in accordance with claim 3, wherein patient or clinician recorded events are used to determine the extent of stimulation required.

19. A method in accordance with claim 3, wherein the step of varying the stimulation comprises varying the stimulation between a plurality of stimulation levels.

20. A method in accordance with claim 19, wherein one of the stimulation levels is absence of stimulation.

21. A method in accordance with claim 20, wherein the absence of stimulation is arranged to enable evacuation of the bladder.

22. A method in accordance with claim 19, wherein one of the stimulation levels is a Low level, arranged to cause coaptation of the urethra with sub-maximal fullness of the bladder.

23. A method in accordance with claim 22, wherein the Low level stimulation is implemented by electrical stimulation having the following parameters: 3 mA to 5 mA current; 0.1 ms to 0.6 ms phase width; 0.5 hZ to 1.5 Hz frequency, of the stimulating signal.

24. A method in accordance with claim 23, wherein the electrical signal has the following parameters: 4 mA, 0.5 ms phase width, 1 Hz frequency.

25. A method in accordance with claim 19, wherein one of the stimulation levels is a Medium level, arranged to cause coaptation of the urethra with greater sub-minimal bladder fullness.

26. A method in accordance with claim 25, Medium stimulation is implemented by an electrical signal having the following parameters: 6 mA to 10 mA current; 0.4 ms to 0.6 ms phase width; 0.5 Hz to 1.5 Hz frequency.

27. A method in accordance with claim 26, wherein the electrical signal has the following parameters: 8 mA current, 0.5 ms phase width, 1 Hz frequency.

28. A method in accordance with claim 19, wherein one of the stimulation levels is a Maximum level arranged to cause coaptation of the urethra with greater than sub-maximal fullness.

29. A method in accordance with claim 28, the Maximum stimulation level being arranged to enable relatively strenuous physical activity of the patient whilst avoiding or minimising bladder leakage.

30. A method in accordance with claim 28, wherein the Maximum stimulation level is implemented by an electrical signal having the following parameters: 6 mA to 10 mA current; 0.4 ms to 0.6 ms phase width; 1.5 Hz to 2.5 Hz frequency.

31. A method in accordance with claim 30, wherein the Maximum electrical signal has the following parameters: 8 mA current, 0.5 ms phase width, 2 Hz frequency.

32. A method in accordance with claim 19, comprising the step of, after a patient voiding event, applying a stimulation level that is relatively low and, after a predetermined time increasing the stimulation level to relatively higher.

33. A method in accordance with claim 32, comprising a further step of applying a relatively high stimulation level prior to applying the relatively low stimulation level.

34. A method in accordance with claim 33, wherein the relatively high stimulation level is applied immediately after a patient voiding event.

35. A method in accordance with claim 19, comprising the further step of logging the time spent in the various stimulation levels.

36. A method in accordance with claim 2, comprising a further step of pre-calibrating a range of stimulation.

37. A method in accordance with claim 36, wherein the step of pre-calibrating comprises the step of starting with minimum stimulation and increasing it until there is a threshold effect.

38. A method in accordance with claim 37, wherein the step of pre-calibrating further comprises the step of applying stimulation and monitoring patient feedback.

39. A method in accordance with claim 36, wherein the step of pre-calibrating includes the step of establishing a plurality of stimulator settings.

40. A method in accordance with claim 39, wherein the step of establishing a plurality of stimulator settings comprises the step of establishing an “Off” setting where there is no or minimal stimulation, enabling voiding of the bladder.

41. A method in accordance with claim 39, wherein the step of establishing a plurality of stimulator settings comprises the step of establishing a Low setting as substantially the minimum stimulation required to maintain continence with sub-maximal bladder fullness.

42. A method in accordance with claim 39, wherein the step of establishing a plurality of stimulator settings comprises the step of establishing a Medium setting as substantially the stimulation required to maintain continence for a range of bladder fullness.

43. A method in accordance with claim 40, wherein the step of establishing a plurality of stimulator settings comprises the step of establishing a High setting as the maximum stimulation intensity without unwanted side effects.

44. A method in accordance with claim 3, wherein the step of determining includes the step of determining a setting of an electrical stimulation setting of a stimulator.

45. A method in accordance with claim 3, where control is implemented by stimulation of a contractile tissue sphincter.

46. A method in accordance with claim 45, wherein the sphincter is arranged to cause closure of the urethra when appropriately stimulated.

47. A method in accordance with claim 46, wherein the sphincter is an implanted smooth muscle sphincter.

48. A method in accordance with claim 45, comprising the further step of applying stimulation to a further anatomical feature as well as the sphincter, in order to augment the affect of stimulation to the sphincter.

49. A method in accordance with claim 48, wherein the additional anatomical feature is the external urinary sphincter.

50. A method in accordance with claim 48, wherein the further anatomical feature is the pelvic floor.

51. A method in accordance with claim 3, wherein the step of determining includes the step of sensing a patient event, and varying stimulation in response to the patient event.

52. A method in accordance with claim 51, wherein the patient event includes one or more of the following, a laugh, a cough, a sudden change in abdominal pressure, physical activity of the patient, posture of the patient.

53-55. (canceled)

56. An apparatus for controlling stimulation of implanted contractile tissue in order to control a bodily function, the apparatus being arranged to determine the extent of stimulation required and to vary the stimulation according to the determination.

57. An apparatus in accordance with claim 56, wherein the implanted contractile tissue is in the pelvic region, for controlling a bodily function in the pelvic region.

58. An apparatus in accordance with claim 57, wherein the bodily function is associated with continence.

59. An apparatus in accordance with claim 58, wherein the bodily function is urinary continence.

60-104. (canceled)

105. A computer program for controlling an implantable stimulator to control stimulation of implanted contractile tissue in order to control a bodily function, in order to implement a method in accordance claim 1.

106. (canceled)

107. A computer readable medium providing a computer program in accordance with claim 105.

108-123. (canceled)
